(19) United States
(12) Patent Application Publication
(10) Pub. No.: US 2002/0165127 A1
(43) Pub. Date: Nov. 7, 2002

(54) Methods for treating insulin resistance and identifying patients at risk for the disease

(75) Inventor: Fred I. Chasalow, San Carlos, CA (US)

Correspondence Address: DARBY & DARBY P.C., 805 Third Avenue, New York, NY, 10022, US

(73) Assignee: SuperGen, Inc.

(21) Appl. No.: 10/079,597

(22) Filed: Feb. 19, 2002

Related U.S. Application Data
(63) Continuation of application No. 09/284,038, filed on Apr. 2, 1999, now abandoned.
Application No. 09/284,038 is a 371 of international application No. PCT/US97/23481, filed on Dec. 23, 1997.
(60) Provisional application No. 60/034,504, filed on Dec. 24, 1996.
(60) Provisional application No. 60/037,417, filed on Feb. 21, 1997.

(51) Int. Cl. A01N 37/18; A61K 38/00; C07K 5/00; C07K 7/00; C07K 16/00; C07K 17/00; A61K 38/28

(52) U.S. Cl. 514/2; 530/303

(57) ABSTRACT

The present invention is directed to compositions and methods for treating insulin resistance and for ameliorating pathological conditions associated with insulin resistance. Also disclosed are methods for identifying patients at risk for developing non-insulin dependent diabetes melitis or insulin resistance. Also disclosed are methods for identifying patients at risk for developing or suffering from reactive hypoglycemia and methods of treatment thereof.

This application claims priority under 35 U.S.C. §119 from Provisional U.S. patent application No. 60/034,504 filed Dec. 24, 1996, and Provisional U.S. patent applicaton No. 60/037,417 filed Feb. 21, 1997, the entire disclosures of which are hereby incorporated by reference.

### FIELD OF THE INVENTION

This invention pertains to methods and compositions for treating insulin resistance and for ameliorating pathological conditions associated with insulin resistance. Also disclosed herein are methods for identifying patients at risk for developing non-insulin dependent diabetes melitis or insulin resistance. The invention also pertains to methods for identifying patients at risk for developing or suffering from reactive hypoglycemia and methods of treatment thereof.

### BACKGROUND OF THE INVENTION

Insulin resistance is an extremely common pathophysiological phenomenon that is implicated in non-insulin-dependent diabetes (NIDDM), atherosclerotic cardiovascular disease, syndrome x, obesity, hypertension, dyslipidemias, and polycystic ovarian syndrome, among other pathological conditions (Moller et al., Diabetes Care 19:396, 1996). At least in some instances, insulin resistance is likely to be caused by competition between insulin and IGF-2 for binding to the insulin receptor, which is present on a large number of different cell types and which mediates the body's response to insulin. This is based in part on the correlation between IGF-2 concentrations and insulin requirements in children with diabetes (Chasalow et al., Amer.Ped.Soc. 1995 meeting). Without wishing to be bound by theory, it is believed that the presence of an increased level of circulating IGF-2 can affect the occupancy of the insulin receptor so as to contribute to insulin resistance; and that, conversely, lowering the circulating levels of IGF-2 in a patient will decrease the magnitude of insulin resistance.

Thus, there is a need in the art for methods to treat insulin resistance that is caused or accompanied by a pathological increase in circulating IGF-2.

### SUMMARY OF THE INVENTION

The present invention encompasses methods for treating insulin resistance in a patient in need of such treatment, which involve: (a) determining the concentration of IGF-2 in the patient's serum and (b) lowering the serum concentration of IGF-2 in the patient. The methods are carried out by administering an agent selected from the group consisting of:

(i) human 20K-GH or pharmacologically active variants or derivatives thereof;

(ii) nucleic acid sequences encoding human 20K-GH or variants or derivatives thereof, in a context that allows cells comprising said nucleic acids to express pharmacologically active 20K-GH or variants or derivatives thereof;

(iii) antibodies specific to human 20K-GH that stabilize 20K-GH in the circulation; and

(iv) combinations of the above.

The agent (which may, in combination with a pharmaceutically acceptable carrier or diluent, comprise a pharmaceutical formulation) is administered in an amount and for a time sufficient to reduce the concentration of IGF-2 in the patient's circulation. Administration may be via any suitable method, including without limitation subcutaneous, intravenous, intramuscular, oral, intranasal, and mucosal modes of administration.

Non-limiting examples of pathological conditions to which the methods of the invention may be applied include non-insulin-dependent diabetes (NIDDM), syndrome X, obesity, polycystic ovarian disorder, hair-AN syndrome, AIDS wasting, intra-uterine growth failure, post-natal growth failure, and Prader-Willi syndrome.

In one embodiment, human 20K-GH produced from a recombinant gene, or a pharmacologically active variant or derivative thereof, is administered in an amount and for a time sufficient to reduce the concentration of IGF-2 in the serum of said patient.

In another embodiment, humanized monoclonal antibodies specific for 20K-GH, which stabilize 20K-GH in the circulation, are administered in an amount and for a time sufficient to reduce the concentration of IGF-2 in the serum of said patient.

In yet another embodiment, the present invention provides a method for identifying a patient at risk for developing insulin resistance comprising the steps of:

(a) determining the amount of 20K-GH in said patients serum; and

(b) comparing the amount of 20K-GH in said patients serum with the amount of 20K-GH present in age and sex matched control serum,

wherein said patient is at risk for developing insulin resistance if the amount of 20K-GH is significantly lower than the amount of 20K-GH present in said control.

In a still further embodiment, the present invention provides a method for identifying a patient at risk for developing insulin resistance comprising the steps of:

(a) determining the amount of IGF-2 in said patients serum; and

(b) comparing the amount of IGF-2 in said patients serum with the amount of IGF-2 present in age and sex matched control serum,

wherein said patient is at risk for developing insulin resistance if the amount of IGF-2 is significantly higher than the amount of IGF-2 present in said control.

In a still further embodiment, the present invention provides a method for identifying a patient at risk for developing reactive hypoglycemia comprising the steps of quantifying the amount of IGF-2 in the patient's serum and comparing the amount with the amount of IGF-2 in age and sex matched controls, wherein the patient is at risk for developing reactive hypoglycemia if the levels of IGF-2 are significantly lower than those present in said control.

In yet a further embodiment, the present invention provides methods for treating patients suffering from reactive hypoglycemia by administering an effective amount for treating reactive hypoglycemia of an agent selected from the group consisting of:

(i) human 22K-GH or pharmacologically active variants or derivatives thereof;

(ii) nucleic acid sequences encoding human 22K-GH or variants or derivatives thereof, in a context that allows cells comprising said nucleic acids to express pharmacologically active 22K-GH or variants or derivatives thereof;

(iii) IGF-2 or nucleic acid sequences encoding IGF-2 or derivatives or variants thereof in a context that allows cells comprising said nucleic acids to express pharmacologically active IGF-2, and

(iv) combinations of the above.

### DETAILED DESCRIPTION OF THE INVENTION

All patent applications, patents, and literature references cited in this specification are hereby incorporated by reference in their entirety. In the case of inconsistencies, the present description, including definitions, will control.

Definitions

1. “20K-GH” refers to the isoform of human growth hormone that has a molecular weight of 20,200 and lacks amino acids 32-46 of the larger human growth hormone isoform, which is designated “22K-GH”. The mRNA encoding, 20K-GH is formed from the human growth hormone gene by an alternative splicing event. The amino acid sequence of 20K-GH is disclosed in Masuda et al., Biochim.Biophy. Acta 949:125, 1988, and that of 22K-GH in Shine et al., Nature 270:494, 1977. As used herein, a “pharmacologically active variant” of 20K-GH is a polypeptide having a sequence related to authentic 20K-GH that is capable of increasing insulin sensitivity in patients to whom it is administered.

2. An “effective treatment” for insulin resistance as used herein is a treatment that results in the lessening or amelioration of at least one symptom of the pathological condition or syndrome that is caused or exacerbated by insulin resistance, or that results in a directly measurable increase in insulin sensitivity in the patient.

The present invention provides a method for treating insulin resistance in humans. According to the invention, a patient who exhibits one or more symptoms of insulin resistance or otherwise suffers from a pathological condition that is caused or exacerbated by insulin resistance is administered a treatment that lowers the serum concentration of IGF-2. In a preferred embodiment, a reduction in the circulating level of IGF-2 is achieved by administration of 20K-GH. Increased insulin sensitivity is believed to result from a lowering of IGF-2.

The invention takes advantage of the different physiological effects of the two primary human GH isoforms and the pharmacokinetics of the IGFs to achieve a reversal of insulin resistance.

(i) Differential effects of GH isoforms: The two GH isoforms, 22K-GH and 20K-GH, appear to differ in their effects on the synthesis and secretion of IGFs. 22K-GH is known to stimulate both IGF-1 and IGF-2. 20K-GH, by contrast, appears to stimulate only IGF-1 and not IGF-2, based on the observation that 20K-GH does not exert the lipolytic effects which are observed with 22K-GH and which are believed to result from GH-mediated stimulation of IGF-2 (Juarez-Aguilar et al., Biochem.Biophys.Res.Comm. 217:28, 1995). As discussed below, the present invention encompasses the administration of 20K-GH to modulate circulating levels of IGF-2.

(ii) IGF pharmacokinetics: IGFs in serum interact with a family of binding proteins designated binding proteins 1-6 (“bp1”-“bp6”). The major IGF binding protein in serum is bp3. Notably, IGF-1 and IGF-2 bind with equal affinity to bp3. Furthermore, binding of an IGF polypeptide to bp3 confers stability on the bound IGF; unbound IGFs have a half-life in serum of about 20 min, while bp3-bound IGFs have a half-life of 24-36 hours. Accordingly, it is believed that, in the presence of a constant serum concentration of bp3, an increase in serum IGF-1 will displace bp3-bound IGF-2. Since the displaced IGF-2 is unstable, displacement of IGF-2 from a bound to a free fraction should result in a decrease in the serum concentration of IGF-2. (See Blackburn et al., Growth Horm.Res.Soc., 1996 Conference, Abstract 0:010, who report the results of a study of transgenic mice that overexpress IGF-2 and/or GH. The authors found that an increase in IGF-2 results in a reduction of circulating IGF-1 levels and concluded that the reduction is “most likely due to the limited serum IGF binding capacity and the rapid clearance of free IGFs”).

As discussed above, at least one possible cause of insulin resistance in humans is an increased level of IGF-2 which competes with insulin for binding to the insulin receptor and thus decreases insulin sensitivity. Accordingly, the methods of the present invention, by lowering circulating IGF-2 levels, promote increased insulin sensitivity/decreased insulin resistance. Any detectable increase in insulin sensitivity is useful, whether by providing direct therapeutic benefit or by allowing the further optimization of treatment regimens.

In practicing the invention, IGF-2 levels are decreased by increasing the circulating levels of IGF-1. This increase in circulating IGF-1 levels is preferably achieved by an indirect method, such as, for example, by treatments that increase the circulating levels of 20K-GH. Such treatments include without limitation administration of any or all of the following: (i) 20K-GH polypeptide or pharmacologically active variants or derivatives thereof; (ii) nucleic acids encoding 20K-GH in a context that allows cells comprising the nucleic acids to express pharmacologically active 20K-GH polypeptide; and (iii) monoclonal anti-20K-GH antibodies. The invention encompasses any treatment that increases the short-term or steady-state concentration of circulating 20K-GH, whether acting primarily by increasing the production of 20K-GH from an endogenous or recombinant gene (such as in methods (i) or (ii) above) or by stabilizing 20K-GH in the circulation (such as in method (iii) above).

20K-GH polypeptide: 20K-GH or a pharmacologically active variant or derivative thereof for use in the present invention may be isolated from its native source or, preferably, from recombinant cells that have been programmed to produce it. U.S. Pat. No. 5,496,713 discloses methods for producing recombinant 20K-GH in E. coli; however, any suitable host cell may be used. Alternatively, 20K-GH or its derivatives may be chemically synthesized by commercially available automated procedures, including, without limitation, exclusive solid phase synthesis, partial solid phase methods, fragment condensation or classical solution synthesis.

In practicing the present invention, many conventional techniques in molecular biology, microbiology, and recombinant DNA, are used. Such techniques are well known and are explained fully in, for example, Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984, (M. L. Gait ed.); Nucleic Acid Hybridization, 1985, (Hames and Higgins); Transcription and Translation, 1984 (Hames and Higgins eds.); Animal Cell Culture, 1986 (R.I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, eds.)

Any method for polypeptide purification well-known in the art may be used to isolate 20K-GH or a derivative, including without limitation preparative disc-gel electrophoresis, isoelectric focusing, HPLC, reversed-phase HPLC, gel filtration, ion exchange and partition chromatography, and countercurrent distribution. Recombinant 20K-GH may also be produced containing an additional amino acid sequence that acts as a “tag” to facilitate purification, such as, but not limited to, a polyhistidine sequence. The 20K-GH can then be purified from a crude lysate or extracellular medium of the host cell by chromatography on an appropriate solid-phase matrix. Alternatively, anti-20K-GH antibodies can be used as purification reagents. Other purification methods are possible. General protein purification methods are described in Protein Purification: Principles and Practice, R. K. Scopes, Ed., 1987, Springer-Verlag, New York, N.Y.

Also encompassed by the invention are pharmacologically active variants of 20K-GH. Natural variants of 20K-GH have been reported, for example, 20K-GH with divergent sequences at codon 14 (Masuda et al., Biochim.Biophys.Acta 949:125, 1988; and Martial et al., Science 205: 602, 1979). Another known variant is Met-20K-GH, which contains an additional methionine residue at the aminoterminus.

20K-GH for use in the invention may also be conjugated to other molecules, such as, for example, polyethylene glycol, fatty acids, and the like. The polypeptide may also be modified by, for example, N-acetylation, deamidation, phosphorylation, sulfation, and the like.

Any variant or conjugate of 20K-GH, including those described above, may be used in practicing the invention, with the proviso that it is pharmacologically active, i.e., mediates an increase in insulin sensitivity in a patient to whom it is administered. A particular 20K-GH variant may be identified as useful in practicing the invention by assessing whether it is capable of mediating one or more of the following activities: an increase in IGF-1, a decrease in IGF-2, or an increase in insulin sensitivity. For example, cultured human cells that secrete both IGF-1 and IGF-2 in response to 22K-GH and secrete only IGF-1 in response to 20K-GH are used to test the suitability of a particular 20K-GH.

20K-GH-encoding nucleic acids: 20K-GH-encoding nucleic acids may be DNA or RNA, and include sense and antisense sequences. The nucleic acids may be flanked by their native regulatory sequences, or may be associated with heterologous sequences, including promoters, enhancers, response elements, signal sequences, polyadenylation sequences, introns, 5′- and 3′-noncoding regions, and the like. The nucleic acids may also be modified by many means known in the art. Non-limiting examples of such modifications include methylation, “caps”, substitution of one or more of the naturally occurring nucleotides with an analog, internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Nucleic acids may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. PNAs are also included.

Preferably, the sequences are present in appropriate nucleic acid vectors operably linked to regulatory sequences that allow their expression in the recipient cells. The expression may be transient or long-term, depending upon the expression system that is utilized. Any appropriate means for delivering the 20K-GH-encoding nucleic acids to the recipient cells may be used, including packaging in liposomes or recombinant viruses. Furthermore, both in vivo and ex vivo approaches may be used. For in vivo approaches, the nucleic acids are introduced directly into the patient; for ex vivo approaches, appropriate recipient cells are first removed from the patient and contacted with the 20K-GH-encoding nucleic acids under conditions appropriate for uptake and expression, after which the transformed cells are re-introduced into the patient.

Monoclonal anti-20K-GH antibodies: An increase insulin sensitivity according to the present invention may also be achieved by increasing the stability of serum 20K-GH and thereby raising its steady-state concentration. For this purpose, any compound may be administered that binds to circulating 20K-GH and retards its degradation, with the proviso that the complex retains the pharmacological activity of 20K-GH in reducing insulin resistance.

In one embodiment, monoclonal antibodies specific for 20K-GH are used. As used herein, antibodies “specific for 20K-GH” recognize and bind to 20K-GH with a sufficient affinity to stabilize it; but, conversely, do not recognize and/or bind to 22K-GH with sufficient affinity to stabilize it. In a preferred embodiment, the antibodies are “humanized”, i.e., human Fc sequences are present in the antibody molecule to prevent an adverse immune response in a patient to whom the antibodies are administered. (See below.)

The general methodology for making monoclonal antibodies by hybridomas is well known. See, e.g. Kohler et al., 1980, Hybridoma Techniques, Cold Spring Harbor Laboratory, New York; Tijssen, 1985, Practice and Theory of Enzyme Immunoassays, Elsevier, Amsterdam; Campbell, 1984, Monoclonal Antibody Technology, Elsevier, Amsterdam; Hurrell, 1982, Monoclonal Hybridoma Antibodies: Techniques and Applications, CRC Press, Boca Raton, Fla. Purification methods for antibodies are disclosed, e.g., in The Art of Antibody Purification, 1989, Amicon Division, W. R. Grace & Co.

The present invention can be applied to any syndrome or disorder that is associated with insulin resistance, low serum levels of 20K-GH, and/or high serum levels of IGF-2, including without limitation non-insulin-dependent diabetes (NIDDM), syndrome X, obesity, polycystic ovarian disorder, hair-AN syndrome, AIDS wasting, intra-uterine growth failure, post-natal growth failure, and Prader-Willi syndrome.

Therapeutic Administration

The therapeutic regimen for using any of the above-described agents for treating insulin resistance can be determined by experimentation known in the art, such as by establishing a matrix of dosages and frequencies and comparing a group of experimental units or subjects to each point in the matrix. It will be understood that the administration regimen (amount and frequency of administration) will vary depending upon the agent itself, the syndrome being treated, and the age, gender, and physical condition of the patient. Any lessening or amelioration of any clinical signs or symptoms of a particular condition or syndrome caused or exacerbated by insulin resistance pursuant to treatment as disclosed herein is within the scope of the invention. Included are any treatments that allow a reduction in the amount and frequency of administration of other drugs used to treat the syndromes, such as, e.g., sulfonylureas, biguanides, α-glucosidase inhibitors, and insulin (Tan et al., Mayo Clin.Proc. 71:763, 1996).

Administration may be via any suitable method, including without limitation subcutaneous, intravenous, intramuscular, oral, intranasal, and mucosal modes of administration. As used herein, “mucosal” administration encompasses the use of suppositories.

In one embodiment, 20K-GH is administered in an effective amount ranging between about 0.0001 and about 0.06 mg/kg/day, preferably between about 0.0005 and about 0.005 mg/kg/day. The dosage and administration regimen of long-acting 20K-GH derivatives is modified to take advantage of this property.

In another embodiment, humanized antibody specific for 20K-GH is administered in sufficient amounts and at sufficient intervals to achieve a detectable increase in circulating 20K-GH. It will be understood that the particular amounts and times of administration will depend upon the particular properties of the antibody being used, including, for example, relative affinities for 20K-GH and 22K-GH and half-life in the circulation.

The agents of the invention may, in combination with a pharmaceutically acceptable carrier or diluent, comprise pharmaceutical formulations. For general information concerning formulations, see, e.g., Gilman et al. (eds.), 1990, Goodmans and Gilman's: The Pharmacological Basis of Therapeutics, 8th Ed., Pergamon Press; and Remington's Pharmaceutical Sciences, 17th ed., 1990, Mack Publishing Co., Easton, Pa.; Avis et al. (eds.), 1993, Pharmaceutical Dosage Forms: Parenteral Medications, Dekker, New York; Lieberman et al. (eds), 1990, Pharmaceutical Dosage Forms: Disperse Systems, Dekker, N.Y.

The invention also encompasses diagnostic tests that are performed to identify patients at risk for developing insulin resistance or non-insulin dependent diabetes melitus (NIDDM) and/or those patients whose pathological conditions are likely to respond to the methods of the invention. Without wishing to be bound by theory, it is believed that serum levels of 20K-GH will be low in individuals suffering from NIDDM or insulin resistance. Thus, an assay for serum levels of 20K-GH can be used to evaluate the risk of a patient developing NIDDM or insulin resistance as set forth below.

The determination involves (i) obtaining a serum sample from the patient; (ii) measuring the absolute amount of 20K-GH in the sample; and (iii) comparing the amount measured in step (ii) with pre-determined levels of 20K-GH in normal subjects matched with the patient with respect to age, gender, etc. If the amount of 20K-GH is significantly lower than that present in the normal subjects, the patient is at risk for developing insulin resistance.

20K-GH and 22K-GH may be measured using any method known in the art, including without limitation immunological methods such as radioimmunoassay, receptor binding assay, physical methods such as gel electrophoresis or chromatography, or combinations of the foregoing. It will be understood that normal levels of 20K-GH and 22K-GH for comparison will be determined using the same assay method, or, in the alternative, that normalization factors will be used to allow a comparison of GH levels.

In a preferred embodiment, antibodies specific for 20K-GH will be generated as follows. As mentioned above, the growth hormone gene codes for the expression of two proteins which differ by an internal 15 amino acid deletion. The cause of the deletion is the expression of an alternate splice in one of the non-translated portions of the gene. In order to generate antibodies specific for 20K-GH, peptides will be selected across the bridge of the deletion (between the Phe and the Agn amino acid residues disclosed below). Non-limiting examples of useful peptides are set forth below in Table I.

TABLE I

Residues

| | |
|---|---|
| NH₂-Tyr-Gln-Glu-Phe-Asn-Pro-Gln-Thr-COOH | 8 |
| NH₂-AsP-Thr-Tyr-Gln-Glu-Phe-Asn-Pro-Gln-Thr-COOH | 10 |
| NH₂-Tyr-Gln-Glu-Phe-Asn-Pro-Gln-Thr-Ser-Leu-COOH | 10 |
| NH₂-Leu-Ala-Phe-Asp-Thr-Tyr-Gln-Glu-Phe-Asn-Pro-Gln- | 15 |
| Thr-Ser-Leu-COOH | |

Each peptide will be coupled to an antigen and used to immunize rabbits for polyclonal antibody production or to mice for monoclonal antibody production. It should be noted that direct administration of 20K-GH to either rabbits or mice yields antibodies that cross-react with both 20K-GH and 22K-GH.

The antibodies can be used to generate an assay for 20K-GH in several different formats. In a preferred embodiment, the peptides will be coupled to thyroglobulin using commercially available reagents (Pierce, Rockford, Ill.) and bound to a 96-well ELISA plate (as described in Steroids 12:682-687, 1996). After the addition of a sample (standard, control or unknown), the antibody would be added, incubated and washed. The amount of antibody remaining on the plate would be quantitated with ELISA techniques. The wells with standards would be used to generate a standard curve and use to evaluate controls and unknowns. In a second embodiment, 20K-GH could be bound to each well. In a third embodiment, the peptides would be radioiodinated, and used as a tracer in a standard RIA format. In a fourth embodiment, 20K-GH could be iodinated and used in an RIA format. In a fifth embodiment, a sandwich assay, the 20K-GH specific antibody could be bound to the well and incubated with standards, controls and unknowns. After washing, a second antibody specific for both 20K-GH and 22K-GH could be added. The amount of the second antibody could be determined by ELISA or by radioiodination of the second antibody.

For use as method of diagnosing risk, data will be generated on the normal values of 20K-GH. This would include evaluating ranges in normal subjects of different ages, sexes, degrees of adiposity, blood pressure and health history. It will also be necessary to evaluate time of day, time of month (women), exercise status, and estimated dietary fat content as variables. Serum obtained from subjects with family histories of NIDDM and newly diagnosed patients would be compared with the normal curve. Other studies to validate the assay will correlate 20K-GH levels with IGF-1, IGF-2, IGF-bp3, hemoglobin AlC, serum glucose levels, and insulin requirements in patients with NIDDM. It is anticipated that the levels of 20K-GH will be significantly low and levels of IGF-2 will be significantly high in patients at risk for the disease. “Significantly low” 20K-GH levels are defined as more than one standard deviation below the mean, “Significantly high” IGF-2 levels are defined as more than one standard deviation above the mean.

In another preferred embodiment of the present invention methods are provided for identifying patients at risk for or suffering from reactive hypoglycemia and methods of treatment thereof. Hypoglycemia is an abnormally low blood glucose level. “Reactive hypoglycemia” is abnormally low blood glucose levels in response to specific factors such as a meal, nutrients, or drugs. It is distinguished from spontaneous hypoglycemia in that it occurs following administration of exogenous factors such as meals (e.g. containing carbohydrates), specific nutrients which inhibit hepatic glucose output, drugs which lead to excess glucose utilization (such as insulin) or those leading to deficient glucose production (alcohol, salicylates, aminobenzoic acid, etc.) as described in The Merck Manual of Diagnosis and Therapy, Vol. 1, 15th Edition, pp. 834-837, R. Berkow, ed. 1987. It has now been unexpectedly discovered that patients suffering from reactive hypoglycemia have significantly lower levels of IGF-2 in their serum. “Significantly lower” is defined herein as greater than one standard deviation below the normal levels determined in age and sex matched controls. Therefore, the present invention provides a method for identifying a patient at risk for or suffering from reactive hypoglycemia comprising the steps of determining the amount of IGF-2 in the patient's serum and comparing the amount of IGF-2 in the patient's serum with the amount of IGF-2 present in age and sex matched control serum, wherein the patient is at risk for reactive hypoglycemia if the amount of IGF-2 present in the patient's serum is significantly lower than the amount of IGF-2 present in the control serum.

In addition, the present invention provides a method for treating reactive hypoglycemia in a patient by administering an agent selected from the group consisting of 22K-GH, a pharmacologically active variant or a derivative thereof in an amount and for a time sufficient to increase the concentration of IGF-2 in the serum of said patient. Alternatively, an effective amount for treating reactive hypoglycemia of IGF-2 or variants thereof can be (directly or indirectly) administered. Direct administration comprises introducing the protein whereas indirect administration comprises introducing a recombinant gene encoding IGF-2. The gene can be administered ex vivo. The DNA and amino acid sequence IGF-2 is disclosed in Rinderknect et al. FEBS Letters 89(2):283-286, 1978. Variants of IGF-2 have been described in FEBS Letters 179: (2);243-246, 1985.

The following examples are intended to serve as non-limiting illustrations of the present invention.

### EXAMPLE 1

### Therapeutic Administration of 20K-GH

A patient suffering from non-insulin dependent diabetes (NIDDM), who currently receives 1 unit/kg/day of intermediate-acting insulin, receives a daily intramuscular administration of a controlled-release formulation containing 20K-GH in sterile phosphate-buffered saline. The patient receives about 0.07 mg 20K-GH per dose, equivalent to a dose of 0.001 mg/kg/day 20K-GH.

The treatment results in a reduction in the amount of insulin that the patient requires on a daily basis to maintain euglycemia.

Another patient suffering from non-insulin dependent diabetes, who currently takes 3.75 mg of Glyburide per day, receives a daily intramuscular injection of controlled release formulation containing 20K-GH in sterile buffered saline. The patient receives about 0.07 mg 20K-GH per dose, equivalent to 0.001 mg/kg/day 20K-GH.

The treatment results in (a) reduction in the amount of Glyburide needed; (b) the patient no longer needs Glyburide to maintain euglycemia or has improved euglycemia control. Therapy need not be on an every day basis.

Another patient with newly diagnosed NIDDM receives a monthly injection of slow release 20K-GH. The treatment results in euglycemia or improved euglycemic control, thus delaying the need for other therapy.

### EXAMPLE 2

### Identification of IGF-2-lowering Variants of 20K-GH

The following experiments are performed to evaluate the suitability of 20K-GH variants or derivatives for use in practicing the present invention. The primary determination is achieved by comparing a particular 20K-GH variant or derivative with authentic 20K-GH itself with respect to the ability to differentially stimulate the release of IGF-1 and not IGF-2 from human cells in culture.

To achieve this aim, a human hepatocyte cell culture such as, e.g., HepG2, is incubated individually with 22K-GH, 20K-GH, and 20K-GH variants in a range of concentrations. The stimulatory effect on IGF-1 and IGF-2 is assessed after different times of incubation by:

(1) quantifying mRNA encoding IGF-1 and IGF-2; and/or

(2) quantifying release of IGF-1 and IGF-2 polypeptides into the culture medium.

Both determinations are performed using methods well-known in the art, such as, for example, Northern blot hybridization (to measure mRNA) and radioimmunoassay (to measure secreted polypeptides).

### EXAMPLE 3

### Treatment of Reactive Hypoglycemia

A patient suffering from reactive hypoglycemia has a serum sample evaluated for IGF-1 and IGF-2 levels. IGF-1 levels are normal but IGF-2 levels are more than two standard deviations below the expected levels for a person of the same age, sex and body weight. The patient is treated by administering IGF-2 or 22K-GH in a long-acting controlled release formulation.

The result of the above treatment is that IGF-2 levels are increased and hypoglycemic episodes become less frequent.

What is claimed is:

1. A method for treating insulin resistance in a patient in need of such treatment, comprising the steps of:
(a) determining the amount of IGF-2 in the serum of said patient; and
(b) lowering the concentration of IGF-2 in the serum of said patient.

2. A method as defined in claim 1, wherein said lowering is achieved by administration of an IGF-2 lowering agent selected from the group consisting of:
(i) human 20K-GH or pharmacologically active variants or derivatives thereof;
(ii) nucleic acid sequences encoding human 20K-GH or variants or derivatives thereof, wherein said nucleic acid sequences are in a context that allows cells comprising said nucleic acids to express pharmacologically active 20K-GH or variants or derivatives thereof;
(iii) antibodies specific to human 20K-GH, wherein said antibodies stabilize 20K-GH in the circulation; and
(iv) combinations of the foregoing;
wherein said agent is administered in an amount and for a time sufficient to reduce the concentration of IGF-2 in the serum of said patient.

3. A method as defined in claim 2, wherein said agent, in combination with a pharmaceutically acceptable carrier or diluent, comprises a pharmaceutical formulation.

4. A method as defined in claim 3, wherein said formulation is administered by a method selected from the group consisting of subcutaneous, intravenous, intramuscular, oral, intranasal, and mucosal modes of administration.

5. A method as defined in claim 1, wherein said patient suffers from a pathological condition selected from the group consisting of: non-insulin-dependent diabetes (NIDDM), syndrome X, obesity, polycystic ovarian disorder, hair-AN syndrome, AIDS wasting, intra-uterine growth failure, post-natal growth failure, and Prader-Willi syndrome.

6. A method for treating insulin resistance in a patient in need of such treatment, comprising administering human 20K-GH, or a pharmacologically active variant or derivative thereof, in an amount and for a time sufficient to reduce the concentration of IGF-2 in the serum of said patient.

7. A method as defined in claim 6, wherein said 20K-GH is produced from a recombinant gene.

8. A method as defined in claim 6, wherein said pharmacologically active 20K-GH variant is Met-20K-GH.

9. A method for treating insulin resistance in a patient in need of such treatment, comprising administering to said patient antibodies specific to human 20K-GH, wherein said antibodies stabilize 20K-GH in the circulation and wherein said antibodies are administered in an amount and for a time sufficient to reduce the concentration of IGF-2 in the serum of said patient.

10. A method as defined in claim 9, wherein said antibodies are monoclonal antibodies.

11. A method as defined in claim 10, wherein said antibodies are humanized antibodies.

12. A method for identifying a patient at risk for developing insulin resistance comprising the steps of:
(a) determining the amount of 20K-GH in said patients serum; and
(b) comparing the amount of 20K-GH in said patients serum with the amount of 20K-GH present in age and sex matched control serum,
wherein said patient is at risk for developing insulin resistance if the amount of 20K-GH is significantly lower than the amount of 20K-GH present in said control.

13. A method for identifying a patient at risk for developing insulin resistance comprising the steps of:
(a) determining the amount of IGF-2 in said patients serum; and
(b) comparing the amount of IGF-2 in said patients serum with the amount of IGF-2 present in age and sex matched control serum,
wherein said patient is at risk for developing insulin resistance if the amount of IGF-2 is significantly higher than the amount of IGF-2 present in said control serum.

14. A method for identifying a patient at risk for developing or suffering from reactive hypoglycemia comprising the steps of
a) determining the amount of IGF-2 present in said patent's serum; and
b) comparing the amount of IGF-2 in said patient's serum with the amount of IGF-2 present in age and sex matched control serum
wherein said patient is at risk for developing or is suffering from reactive hypoglycemia if the amount of IGF-2 in said patient's serum is significantly lower than the amount of IGF-2 present in said control serum.

15. A method for treating a patient suffering from reactive hypoglycemia, comprising administering to a patient in need of such treatment, an agent selected from the group consisting of 22K-GH, a pharmacologically active variant or derivative thereof in an amount and for a time sufficient to increase the concentration of IGF-2 in the serum of said patient.

16. A method as defined in claim 15 wherein said 22K-GH is produced from a recombinant gene.

17. A method as defined in claim 15 wherein said gene is administered ex vivo.

18. A method as defined in claim 15 wherein the agent is IGF-2.

19. A method as defined in claim 18 wherein IGF-2 is produced by a recombinant gene.

20. A method as defined in claim 18 wherein said gene is administered ex vivo.

21. A method for treating a patient suffering from insulin resistance comprising administering to a patient in need of such treatment an effective amount for treating insulin resistance of 20K growth hormone.
